(19) United States
(12) Patent Application Publication
(10) Pub. No.: US 2017/0297003 A1
(43) Pub. Date: Oct. 19, 2017

(54) BIPHASIC SUPERABSORBENT MATERIAL AND DERIVED USES THEREOF

(71) Applicant: MATTHEWS INTERNATIONAL S.p.A., COLORNO (IT)

(72) Inventors: Michele TOSTI, San Casciano In Val Pesa (IT); Luciana SARTORE, Marano Vicentino (IT)

(21) Appl. No.: 15/515,619

(22) PCT Filed: Sep. 30, 2015
(86) PCT No.: PCT/IB2015/057475

§ 371 Date: Mar. 30, 2017

(30) Foreign Application Priority Data
Sep. 30, 2014 (IT) MI2014A001709

(51) Int. Cl. B01J 20/26; A61L 15/22; A61L 15/60; A61G 17/047; B01D 15/08; B01J 20/28; C02F 1/28; C08L 67/04; C08J 5/18; A61L 15/62

(52) U.S. Cl. CPC B01J 20/267; C08J 5/18; A61L 15/225; A61L 15/60; A61L 15/62; B01D 15/08; B01J 20/28033; C02F 1/285; C08L 67/04; A61G 17/047; C08J 2367/04; C08J 2433/02; C08J 2433/26; A61L 2400/04; C08L 2201/06; C08L 2203/16; C08L 2205/22

(57) ABSTRACT

The present invention relates to a biphasic superabsorbent and partially biodegradable material comprising a biodegradable and/or compostable polymeric phase and a non-biodegradable superabsorbent cross-linked polymer phase. Furthermore, the present invention relates to a process for the production of said superabsorbent material and the use thereof to contain and/or absorb and/or separate liquids, in particular aqueous liquids, preferably contaminated ones, and/or biological fluids.

The present invention relates to a biphasic superabsorbent and partially biodegradable material comprising a biodegradable and/or compostable polymeric phase and a non-biodegradable superabsorbent cross-linked polymer phase. In particular, the biodegradable and/or compostable polymeric phase is grafted on non-biodegradable superabsorbent cross-linked polymer particles.

Furthermore, the present invention relates to a process for the production of said superabsorbent material and the use thereof for containing and/or absorbing and/or separating liquids. In particular, the material of the present invention can be used to contain and/or absorb liquids, preferably contaminated liquids and/or biological fluids, for example the ones released during the decomposition of a body.

With a view to preventing/containing the release of contaminated liquids or potentially hazardous biological fluids into the environment, researchers are constantly seeking new materials that have an improved absorbent capacity and at the same time are safe for the environment and human health.

In general, the subject of liquid absorption has been broadly addressed by the scientific community; however, the solutions proposed regard materials that are capable of absorbing liquids, not of retaining and containing them. These are usually materials consisting of unprocessable particulate solids that transform a liquid into a gel.

At present, there exist no films, bags, bottles or objects obtainable in the desired shapes and sizes and capable of containing and absorbing a liquid.

As a solution to the technical problems connected with the containment and/or absorption of liquids, preferably contaminated liquids and/or biological fluids, the Applicant proposes the biphasic superabsorbent and partially biodegradable material of the present invention, said material comprising a biodegradable and/or compostable polymeric matrix grafted on non-biodegradable superabsorbent cross-linked polymer particles.

Unlike the materials presently available, the material of the present invention maintains the workability typical of polymeric materials thanks to its biphasic morphology. In addition, in contact with liquids the material of the present invention forms a barrier capable of retaining them for a substantial amount of time. In particular, when the material of the present invention interacts with aqueous liquids, e.g. natural water, water containing contaminants or biological fluids, it swells immediately; in particular, thanks to their high absorbent power and consequent increase in volume, the dispersed particles rich in superabsorbent polymer are released by the matrix and once released can absorb huge amounts of liquid, converting it into a gel.

The remaining matrix on which the particles are grafted continue to play a role of (structural) containment for long periods of time.

Advantageously, this material is generally capable of very effectively absorbing and/or retaining large amounts of liquids and/or fluids, while at the same time blocking them, i.e. containing them.

Given its noteworthy absorbent capacities, the material of the present invention can have different applications; for example it can be used:
To limit and manage flood phenomena and/or discharges of liquids due to accidental causes, to restore and/or reclaim land, or package organic material;
In emergency situations, to contain and/or isolate percolation phenomena involving polluted waters, for example water containing plant protection products, heavy metal ions, surfactants, fuels or oils;
To separate liquids, for example water/hydrocarbons (for example petrol, petroleum etc.) or water/oils; and
In the biomedical sector to contain and/or stop leakage of blood, for example in haemophilic patients or in haemorrhage emergencies.

The Applicant has found using the absorbent material of the present invention to be particularly useful inside a coffin, for example as a replacement for a galvanized metal case. In this context, the material of the present invention is capable of speeding up the process of decomposition of the body without generating malodorous substances in the surrounding environment. Moreover, it facilitates disposal of the coffin when the body remains are removed.

The advantages of the superabsorbent material of the present invention will become more apparent in light of the detailed description that follows, also with the aid of the accompanying figures, in which:

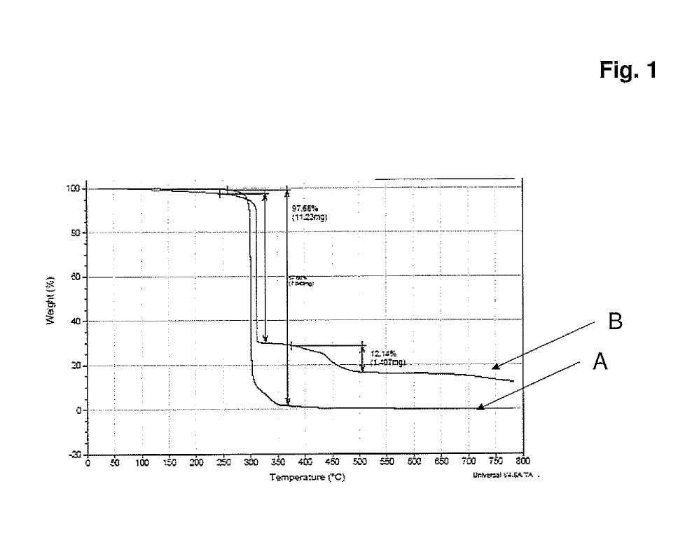

FIG. 1 shows a thermogravimetric analysis of the sample of example 3 (curve B) and of the polymeric matrix (curve A);

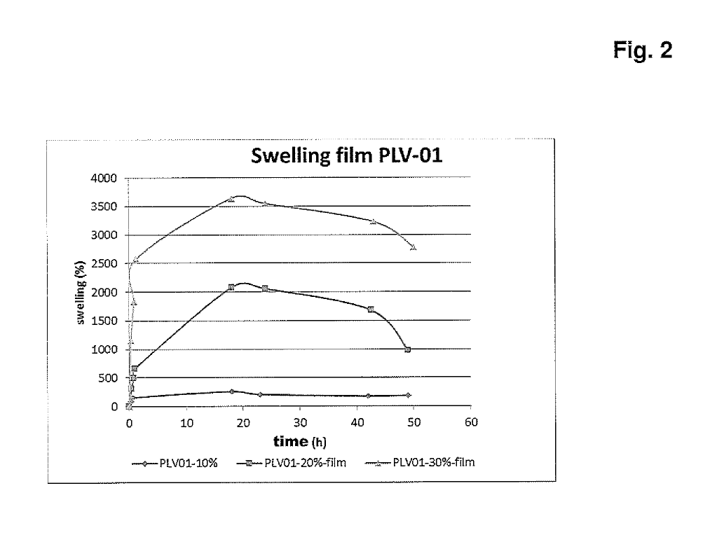

FIG. 2 shows the absorption profile for mains water, as a function of time, of films obtained from the materials of examples 1-3;

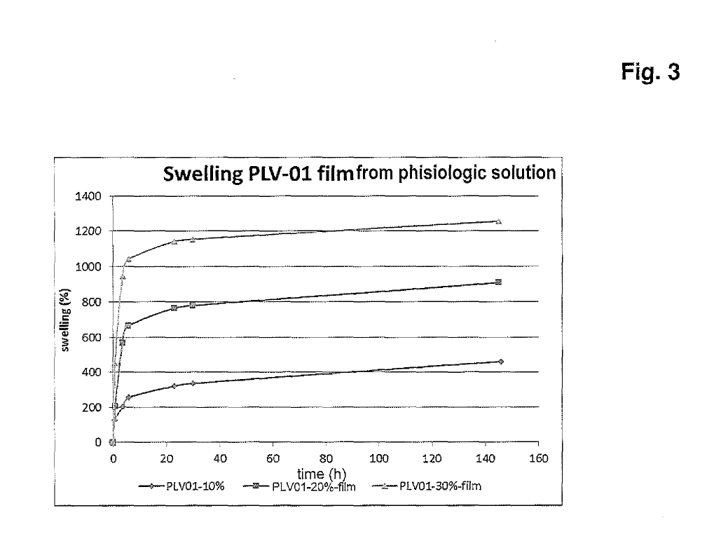

FIG. 3 shows the absorption profile for a saline solution, as a function of time, of films obtained from the materials of examples 1-3,

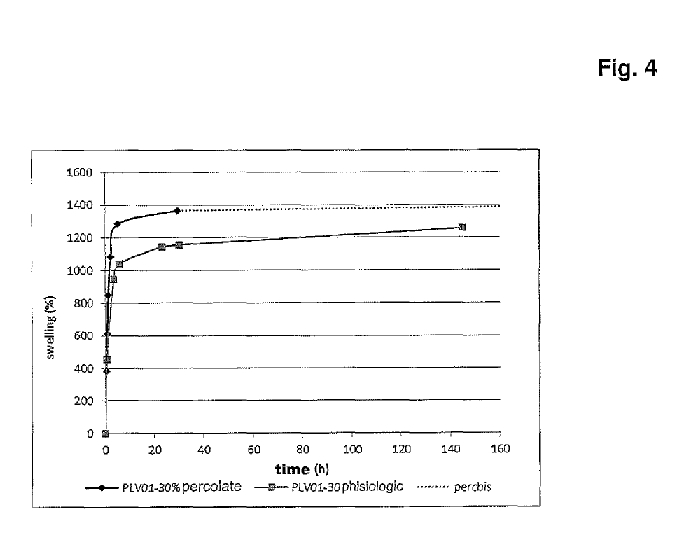

FIG. 4 shows the absorption profile of bodily fluids expelled from a corpse (black curve) and saline solution (grey curve), as a function of time, of films obtained from the materials of example 3.

In a first aspect, the present invention relates to a biphasic superabsorbent and/or partially biodegradable material comprising a biodegradable polymer phase and a non-biodegradable superabsorbent cross-linked polymer phase. In particular, the material of the present invention is preferably thermoplastic and preferably comprises a biodegradable polymer matrix that is grafted, preferably by means of covalent bonds, on particles of a non-biodegradable superabsorbent cross-linked polymer.

The covalent bonds are preferably formed following transesterification reactions. Therefore, the biodegradable and/or compostable polymer is partially grafted onto the non-biodegradable superabsorbent cross-linked polymer particles.

The non-biodegradable superabsorbent cross-linked polymer particles are evenly distributed on the biodegradable and/or compostable polymer. Said particles have sizes preferably ranging from 1 micron (0.1 μm) to 2 mm, more preferably ranging from 1 to 60 μm, and even more preferably 5-10 μm.

The biodegradable and/or compostable polymer according to the present invention is preferably a polymer selected from among: polylactic acid (PLA), poly lactic/glycolic acid (PLGA), polycaprolactone (PCL), polyvinyl acetate (PVA) and polyvinyl alcohol (PVOH), and copolymers or blends thereof (polymer mixture).

Preferably, the polymer used is PLA. For the purposes of the present invention, use can be made of the L isomer of PLA alone or the D isomer alone, or else a blend of L and D isomers, or a racemate of PLA.

Preferably, the viscosimetric molecular weight of the PLA (Mv) ranges between 2 and 400 kDa, more preferably it ranges between 10 and 200 kDa, and even more preferably it ranges between 10 and 80 kDa.

The superabsorbent polymer (SAP) is preferably a polyacrylic acid or polyacrylamide homopolymer. Alternatively, the superabsorbent polymer is a copolymer, preferably of acrylic acid and acrylamide.

Preferably, the superabsorbent polymer has a polyelectrolyte structure and, optionally, it is salified partly or completely. The preferred forms for the purposes of the present invention are those salified with Na, K, Ca or Mg.

The amount of biodegradable polymer ranges between 20 and 95%, preferably it ranges between 40 and 90%, and more preferably it ranges between 40 and 80%. The amount of superabsorbent polymer ranges between 1 and 50%, preferably it ranges between 10 and 40%.

In a particularly preferred embodiment of the invention, the superabsorbent material comprises 10-40% of superabsorbent polymer and 60-90% of biodegradable polymer. The percentages are expressed in weight.

In one embodiment of the present invention, the superabsorbent material preferably also comprises a filler and/or additive capable of modulating the mechanical and/or technological properties and/or the durability and the aesthetic appearance of the material.

For example, fillers and/or natural fibres can be added, e.g. fibres deriving from cotton, linen, hemp, jute, ramie, sisal, coir, fibres of carbon, cellulose, hemicellulose or lignin. Or else lignocellulosic fibres, wood flour or active carbon can be added.

Alternatively, inorganic fillers characterized by a variable mechanical and/or morphological resistance and/or stiffness can be added. Preferably, the inorganic fillers are selected from among: calcium carbonate, calcium sulphate, talc, silica, glass fibres and clayey minerals.

In some embodiments, the superabsorbent material of the present invention can optionally further comprise substances commonly used in the industrial realm for the purpose of improving mixing, extrusion, film coating processes or printability and/or for the purpose of modifying the morphology of the materials, for example: plasticizers, curing agents, thickeners and dispersants.

In a further embodiment of the present invention, the superabsorbent material optionally further comprises pigments and/or colourants.

These further components are preferably added to the blend of biodegradable polymer and cross-linked superabsorbent polymer during the process of producing the material.

A second aspect of the present invention relates to the process for the production of the superabsorbent material which comprises having the biodegradable and/or compostable polymer react with particles of non-biodegradable superabsorbent cross-linked polymer in the state of polymer melt and preferably in the absence of solvents. More preferably, the reaction takes place in the presence of catalysts.

In the context of the present invention, state of polymer melt means a high viscosity fluid that is obtained by heating polymers to a high temperature.

In order to increase the reactivity between the functional groups of the biodegradable polymer and the superabsorbent particles it is further possible to add transfer agents, transesterification catalysts or oxidants to the reagents.

Preferably, the transfer agent is selected from among: tetrabutylammonium tetraphenylborate, tin (II)-2-ethylhexaneate and N-acetyl-epsilon caprolactone.

The oxidant is preferably dicumyl peroxide.

The above-described reaction step can be carried out in a discontinuous mixer or in a twin-screw extruder at a temperature preferably ranging from 150 to 200° C., more preferably from 165 to 175° C. Preferably, the ratios in quantitative terms between the biodegradable polymer and the cross-linked superabsorbent polymer in the blend are the ones given above.

By means of said process one obtains the biphasic superabsorbent material of the present invention, which is capable of effectively absorbing liquids in large amounts, in particular liquids, preferably contaminated liquids, and/or biological fluids. Therefore, the subject matter of the present invention further relates to a biphasic superabsorbent and partially biodegradable and/or compostable material obtained/obtainable with the above-described process.

In the context of the present invention, biological fluids means the liquids and/or materials of human and/or animal origin comprising excreta, secretions, blood (haemoderivatives), tissues or fluids that can potentially cause infections, allergies and intoxications in the exposed individual, for example: blood, urine, sputum, vomit, vaginal secretions, seminal fluid, pleural fluid, peritoneal fluid, pericardial fluid, amniotic fluid, synovial fluid or cerebrospinal fluid.

In particular, the superabsorbent material of the present invention is capable of absorbing liquids and at the same time of acting as a barrier, i.e. of blocking the same, in particular of blocking leachates. Therefore, the material of the present invention defines a superabsorbent and partially biodegradable means or structure which simultaneously absorbs and retains the liquids; in particular it contains leachates.

In this regard, the biphasic composition of superabsorbent material of the present invention, i.e. the biodegradable polymer and particles of a non-biodegradable superabsorbent cross-linked polymer, proves to be particularly advantageous. In fact, the presence of non-biodegradable superabsorbent cross-linked polymer particles within a biodegradable polymer matrix enables the functional properties of the superabsorbent material to be maintained even when the biodegradable polymer matrix is naturally degraded, for example by external agents and/or microorganisms such as fungi and bacteria.

The Applicant has found that the two polymers processed under the conditions described in the present invention are capable of generating exchange reactions, in particular transesterification reactions, thus creating real covalent bonds between the two phases. Such bonds enable the biodegradable polymer to be grafted on the non-biodegradable superabsorbent cross-linked polymer particles.

Grafting the polymer on the particles considerably influences the physicochemical and mechanical properties of the material obtained, namely, the superabsorbent material of the present invention. In fact, melt blending makes it possible to achieve a first chemical compatibilization, characterized by the formation of covalent bonds, thanks above all to transesterification reactions, and subsequently a physical compatibilization enabling sole adhesion between the grafted particles, the polymer and any other dispersed particles. In particular, melt blending enables reactivity among the components: in fact, at the end of the process one obtains a homogeneous distribution of particles which are partly linked to the matrix by means of covalent bonds and cannot be separated or extracted therefrom. Following their contact with the liquids, the grafted particles will not be released until degradation phenomena arise. Thanks to the covalent grafting of the particles, the absorbent material of the present invention exhibits optimal mechanical properties. In particular, the superabsorbent polymer particles act as reinforcement and indeed the elastic modulus of the material increases proportionately to the amount of superabsorbent polymer particles.

A further advantageous feature of the superabsorbent material according to the present invention is the partial compostability and/or biodegradability thereof. In other words, its use does not create any disposal problems.

The physical characteristics of the superabsorbent material according to the present invention largely depend on the polymeric matrix used.

When the polymer is PLA, the superabsorbent material of the present invention is characterized by a glass transition temperature (Tg) preferably ranging from 50 to 75° C., more preferably from 55 to 70° C., and even more preferably from 60 to 68° C.

Advantageously, said material is stable up to over 300° C.

The stiffness of the superabsorbent material according to the present invention also largely depends on the polymeric matrix used and, preferably, the stiffness of said material increases with increasing amounts of superabsorbent polymer particles in the final compound.

The absorption capacity of said material is very high and depends on the liquids absorbed. In particular, in the case of water absorption, the absorption capacity of the material is equal to about 3500%.

The superabsorbent material of the present invention can be further processed in order to obtain articles or products. In particular, it can be processed, for example, by means of the common techniques used for that purpose, such as compression moulding, injection moulding or blow moulding, or else by calandering and/or thermoforming.

Therefore, the subject matter of the present invention further relates to an article or product comprising at least one layer of the superabsorbent material of the present invention. Preferably, the article or the product is selected from among: a membrane or multilayer film comprising at least one layer of the absorbent material of the present invention. Alternatively, said article or product is a container means such as a bag or a bottle. In particular, the membrane can comprise at least one layer of absorbent material according to the present invention and one or more layers characterized by a different composition and functionality. Such layers can consist of permeable biodegradable materials and/or be alternated with superabsorbent film with the aim of modulating the rate of absorption and the total amount of liquid absorbed. For this purpose, for example, use can be made of sheets of paper, cardboard, sheets of cellulose, cotton or nonwoven fabric.

In some embodiments, use can also be made of layers of another polymer selected, for example, on the basis of the required functionality. In this manner it is possible to compose a proper multilayer film.

The Applicant has found it particularly advantageous to use the superabsorbent material of the present invention as an absorption means/structure to be placed inside a coffin for corpses, in particular, as a replacement for the galvanized metal cases normally used to preserve corpses.

Therefore, the subject matter of the present invention further relates to a case, preferably sealed, comprising at least one layer of material according to the present invention.

According to one embodiment, the superabsorbent material of the present invention is used for the purpose of limiting and/or managing flood phenomena and/or discharges of liquids due to accidental causes, or restoring and/or reclaiming land.

Alternatively, the superabsorbent material of the present invention is used to package organic material or for the purpose of containing and/or isolating percolation phenomena involving polluted waters, containing, for example, plant protection products, heavy metal ions, surfactants, fuels or oils.

According to a further embodiment of the present invention, the superabsorbent material is used for separating liquids, for example water/hydrocarbons.

Furthermore, the superabsorbent material of the present invention can be used in the biomedical sector, preferably for the purpose of containing and/or stopping leakage of blood, for example in haemophilic patients or in haemorrhage emergencies.

Some embodiments of the present invention are described below solely by way of non-limiting example.

### EXAMPLES 1-5

### Obtainment of Biphasic PLA-SAP Based Materials

Variable amounts of polylactic acid (PLA) and superabsorbent polymer (SAP) were made to undergo bulk reaction, in the state polymer of melt, without the addition of solvents, and with the optional addition of a transesterification agent, by means of a Brabender-type discontinuous mixer with 2 converging rotors.

The temperature was set on 170° C.; the initial rotor speed was 30 rpm for 2 minutes, then 50 rpm for 5 minutes.

The amounts of reagent used and the products obtained are shown in Table I.

TABLE I

| | | | | | W tot |
|---|---|---|---|---|---|
| | | PLA | SAP | CB | [g] |
| Example 1 | [g] | 49.04 | 6.16^{a} | — | 55.20 |
| V01-10% | [%] | 88.84 | 11.16 | | |
| Example 2 | [g] | 43.72 | 12.85 ^{a} | — | 56.57 |
| V01-20% | [%] | 77.28 | 22.72 | | |
| Example 3 | [g] | 39.58 | 18.03 ^{a} | — | 57.61 |
| V01-30% | [%] | 68.70 | 31.30 | | |
| Example 4 | [g] | 39.24 | 18.36 ^{b} | — | 57.60 |
| V04-30 | [%] | 68.13 | 31.88 | | |
| Example 5 | [g] | 37.07 | 18.04 ^{a} | 2.15 | 57.26 |
| V01-CB | [%] | 64.74 | 31.50 | 3.75 | |

^{a} The SAP used is a cross-linked sodium polyacrylate.
^{b} The SAP used is a cross-linked acrylamide/acrylic acid copolymer salified with potassium.

The materials obtained were of excellent quality and apparently homogeneous. No embrittlement was noted, nor any yellowing observed, and any charges (carbon black) were homogeneously distributed in the material.

The materials obtained were also processed with a twin-screw extruder and then compression moulded to obtain samples and films of varying size and thickness.

In short, they showed excellent workability, comparable to that of polymeric materials.

### Thermal Characterization of the Materials.

A thermal analysis was performed on the materials using differential scanning calorimetry (DSC) and thermogravimetry (TGA).

The DSC technique made it possible to determine such calorimetric parameters as glass transition temperature (Tg), melting temperature of the crystalline phase (Tm) and percent crystallinity (X).

The parameters measured are shown in Table II.

Thermogravimetric analysis (FIG. 1) showed that the polymeric matrix is thermally stable up to over 300° C. (curve A), whilst in the final material (curve B), the grafted particles contributed to increasing this thermal resistance.

TABLE II
1st Heating

| | Tg | DCp | Tc | DHc | Tm | DHm | |
|---|---|---|---|---|---|---|---|
| Sample | (° C.) | (J/g K) | (° C.) | (J/g) | (° C.) | (J/g) | X (%) |
| PLA 2003D | 65.10 | 0.461 | | | 152.06 | 32.60 | 34.82 |
| PLA 2002D | 65.23 | 0.285 | | | 153.98 | 33.12 | 35.38 |
| Example 1 V01 10 | 61.22 | 0.481 | 119.70 | 10.57 | 155.51 | 18.17 | 8.12-9.1 |
| Example 2 V01 20 | 60.88 | 0.444 | 125.53 | 10.07 | 154.23 | 11.78 | 1.8-2.33 |
| Example 3 V01 30 | 60.54 | 0.385 | 129.16 | 7.45 | 155.55 | 7.09 | 0 |

### Mechanical Characterization of the Materials—Tensile Test.

The materials of examples 1, 2 and 3 were subjected to a tensile test in order to determine their mechanical characteristics.

The results obtained, shown in Table III, are typical of polymeric materials with high resistance and high stiffness.

Moreover, stiffness, as represented by the elastic modulus, increases with increasing amounts of SAP particles in the final compound.

TABLE III

| | E (MPa) | ε_{b} (%) | σ_{b} (MPa) |
|---|---|---|---|
| PLA2002 | 3020 | 3.3 | 59.2 |
| Example 1 | 3380 | 2.3 | 47.6 |
| V01-10 | | | |
| Example 2 | 3830 | 1.4 | 37.3 |
| V01-20 | | | |
| Example 3 | 4270 | 1.1 | 31.2 |
| V01-30% | | | |

### Absorption Tests on the Materials.

The materials prepared in the previous examples absorb considerable amounts of liquid of varying kind and swell.

FIGS. 2-4 shows the tests of swelling over time obtained from the materials described in examples 1-5 carried out, respectively, on mains water, saline solution and bodily fluid expelled from a corpse.

These tests demonstrated that the new materials have a very high absorption capacity, which manifests itself above all from the first moments of contact with aqueous liquids.

After the first 5-10 hours have elapsed, depending on the liquids considered, the absorption curves reach a nearly stationary state.

The percent absorption value in this zone for the materials of examples 3, 4 and 5 is shown in Table IV.

TABLE IV

| | | | Max Sw. (%) |
|---|---|---|---|
| | Max Sw. (%) | Max Sw. (%) | expelled |
| | mains water | saline | bodily fluid |
| Example 3 | 3500 | 1250 | 1360 |
| V01-30 | | | |
| Example 4 | 3500 | 1400 | — |
| V04-30 | | | |
| Example 5 | — | 1430 | — |
| V01-CB | | | |

1. A biphasic superabsorbent and partially biodegradable and/or compostable material comprising a biodegradable and/or compostable polymeric matrix phase and a phase of non-biodegradable superabsorbent cross-linked polymer particles, said superabsorbent cross-linked polymer particles being grafted preferably by means of a covalent bond on said biodegradable and/or compostable polymer.

2. The material according to claim 1, wherein the biodegradable polymer is selected from among: polylactic acid (PLA), poly lactic/glycolic acid (PLGA) polycaprolactone (PCL) polyvinyl acetate (PVA), polyvinyl alcohol (PVOH), and copolymers or blends thereof.

3. The material according to claim 1, wherein the biodegradable polymer has a viscosimetric molecular weight (Mv) ranging between 2 and 400 kDa, more preferably it ranges between 10 and 200 kDa, and even more preferably between 10 and 80 kDa.

4. The material according to claim 1, wherein the superabsorbent polymer (SAP) is a polyacrylic acid or polyacrylamide homopolymer, or a copolymer of cross-linked acrylic acid and acrylamide.

5. The material according to claim 1, wherein the superabsorbent polymer has a polyelectrolyte structure and is preferably salified partly or completely.

6. The material according to claim 1, wherein the amount of biodegradable and/or compostable polymer ranges between 20 and 95%, preferably it ranges between 40 and 90%, more preferably it ranges between 40 and 80%.

7. The material according to claim 1, wherein the amount of superabsorbent polymer ranges between 1 and 50%, preferably it ranges between 10 and 40%.

8. The material according to claim 7, wherein the amount of superabsorbent polymer ranges between 10 and 40% and the amount of biodegradable polymer ranges between 60 and 90%.

9. The material according to claim 1, wherein the superabsorbent material further comprises a filler and/or additive that is capable of modulating the mechanical and/or technological properties of said material.

10. The material according to claim 1, wherein the superabsorbent material further comprises a pigment.

11. A process for the production of the material according to claim 1 comprising at least one phase of having a biodegradable and/or compostable polymer react with particles of non-biodegradable superabsorbent cross-linked polymer in the state of polymer melt, in the absence of solvents, and preferably with the addition of catalysts.

12. An article or product comprising at least one layer of material according to claim 1.

13. A membrane comprising at least one layer of material according to claim 1.

14. A case, preferably a sealed case, comprising at least one layer of material according to claim 1.

15. Use of the material according to claim 1 for absorbing and/or containing liquids, preferably contaminated liquids and/or biological fluids, or for limiting and/or managing flood phenomena and/or discharge of liquids owing to accidental causes, or for restoring and/or reclaiming land, or for packaging organic material, or for containing and/or isolating percolation phenomena involving polluted waters, or for separating liquids, preferably water/hydrocarbons, or for containing and/or stopping leakage of blood.
